(19) United States
(12) Patent Application Publication
(10) Pub. No.: US 2009/0297502 A1
(43) Pub. Date: Dec. 3, 2009

(54) CCR2 ANTAGONISTS FOR CHRONIC ORGAN TRANSPLANTATION REJECTION

(76) Inventors: Li Li, Downingtown, PA (US); Sicco H. Popma, Chalfont, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/302,427

(22) PCT Filed: Jun. 14, 2007
(86) PCT No.: PCT/US07/71163

§ 371 Date: Aug. 6, 2009

Related U.S. Application Data
(60) Provisional application No. 60/813,960, filed on Jun. 15, 2006.

(51) Int. Cl. A61K 39/395; A61K 38/13; A61K 31/436; A61K 31/4355; A61K 31/573

(52) U.S. Cl. 424/130.1; 514/11; 514/219; 514/177

(57) ABSTRACT

Anti-chemokine monoclonal antibody therapy is provided for the prevention, control or reversal of chronic rejection mediated vascular remodeling, including, e.g., but not limited to MCP-1/CCR2 antagonist antibody therapy for the modulation of cardiovascular pathologies associated with cardiac graft rejection including intimal thickening, arteritis, fibrosis, and necrosis.

### PRIOR APPLICATION

This application claims priority to U.S. application No. 60/813,960, filed Jun. 15, 2006, which is entirely incorporated herein by reference.

### BACKGROUND OF THE INVENTION

1. Field of the Invention

The invention relates to methods of the using and antagonist of CCL2 binding to CCR2, such as an anti-CCL2 antibody, for the prevention and control of chronic rejection in allograft transplantation.

2. Description of the Related Art

With improved immunosuppression to control acute rejection, the short-term success rate of organ allografting has increased dramatically. However, the half-life of the transplant has remained more or less the same for the past 15 years. In renal transplantation, approximately half of the graft loses are due to chronic allograft rejection (Hayry, Transplant Proc 31 (suppl 7A): 5S, 1999). Currently, there is no drug that can control or reverse chronic rejection; therefore, chronic rejection has emerged as the major unmet medicate need for organ transplantation (Waaga, et al, Curr Op Immunol 12: 517, 2000).

The pathogenesis of chronic rejection is not well understood. Unlike acute rejection, where the dominant histologic feature is lytic with high levels of activation of lymphocytes and inflammatory cells causing tissue destruction, the dominant histologic feature is proliferative. Prominent features include persistent and often low-level perivascular inflammation (arteriosclerosis) of graft arteries and interstitial fibrosis (Hayry, 1999 supra). In the affected graft arteries vascular lumen is replaced by an accumulation of smooth muscle cells and connective tissues in the vessel intima resulting in luminal occlusion. Graft arteriosclerosis is frequently seen in failed cardiac and renal allografts and can develop in any vascularized organ transplant within 6 months to a year after transplantation (Abbas, et al. Chronic Rejection, In: Cellular and Moleular Immunology, 5^{th} Ed., P. 283, 2005).

The neointimal proliferation and fibrosis of chronic rejection is associated with immune-mediated inflammatory reactions and production of cytokines and chemokines (Libby, 2001 Immunity, 14:387). The first stage in fibrogenesis associated with chronic rejection or other fibrogenic mechanisms is the recruitment and activation of monocyte/macrophage, which results in the production of proinflammatory mediators (TGFβ, TNFα, IL-16, etc.), which recruits and stimulates the proliferation of fibroblasts (Eugui, 2002 Transplantation Proceedings, 34: 2867).

Monocyte chemoattractant protein 1 (MCP-1, CCL2, ligand for CCR2, GenBank NP_{—}002973), an 8.6 kDa protein containing 76 amino acid residues, is a member of the chemokine-beta (or C-C) family of cytokines. MCP-1 is expressed by a variety of cell types including monocytes, vascular endothelial cells, smooth muscle cells, glomerular mesangial cell, osteoblastic cells, and human pulmonary type-2-like epithelial cells (Sanders, S K. et al. Journal of Immunology, 165: 4877-4883, 2000). It is believed that MCP-1 plays an active role in the initiation and progression of inflammatory diseases, by promoting monocyte influx and subsequent activation in tissues. MCP-1 is chemotactic for monocytes but not neutrophils. It can induce the proliferation and activation of killer cells known as CHAK (CC-chemokine activated killer), which are similar to cells activated by IL-2. It regulates the expression of cell surface antigens (CD11c, CD11b) and the expression of cytokines IL1 and IL6. MCP-1 is a potent activator of human basophils, inducing the degranulation and the release of histamines.

Thus, there is a need in the art of transplantation to enhance the safety and survival in allograft utilization and to understand and remedy the pathological actions of MCP-1 therein.

### SUMMARY OF THE INVENTION

The present invention provides a method of preventing, slowing, or reversing the vascular pathology related to chronic rejection of a transplanted tissue in a mammalian subject, comprising administering to said subject a therapeutically effective amount of an CCR2 antagonist. In one aspect of the invention the subject is human receiving a cardiac allograft.

The method of the invention may be practiced with a CCR2 antagonists which prevents the biological functions or bioactivity associated with CCR2, its isoforms or variants including CCR2A or CCR2B, in cells that display the receptor as defined herein. In one aspect of the invention, CCR2 antagonists include antibodies, synthetic or native sequence peptides and small molecule antagonists, which bind MCP-1/CCL2 or CCR2 or which prevent the binding of CCR2 with its cognate ligand(s) and thereby inhibit CCR2 biological functions.

### BRIEF DESCRIPTION OF THE SEVERAL VIEWS OF THE DRAWING

FIG. 1 is a survival graph showing the time of survival post-transplantation of C57/B6 (H-2b) mice having received heterotopic heart transplant from C3H(H-2k) mice, and treated with anti-JE (C1142) or an irrelevant control mAb (CNTO1322) at 1 mg/mouse i.p. on days 0, 1, 3, 5 and 7.

FIG. 2 are H & E stained vessels analyzed for vascular intimal thickening from C57/B6 (H-2b) mice having received heart transplant from C3H(H-2k) mice, treated with a standard short course of anti-CD45RB to prevent acute rejection, and at one month given either anti-JE or control Ab (1 mg/mouse i.p.) on days 0, 1, 3, 5 and 7): A-1 and A-2 are two representative grafts from the anti-CD45RB only treated group with the arrows defining the width vascular intima, while B-1 and B-2 from the anti-CD45RB plus anti-JE treated group.

FIG. 3 are the same representative tissue sections as in FIG. 2 stained with Trichrome in order to visualize collagen deposition which represents fibrosis from C57/B6 (H-2b) mice having received heart transplant from C3H(H-2k) mice, treated with a standard short course of anti-CD45RB to prevent acute rejection, and at one month given either anti-JE or control Ab (100 μg i.p. injection twice weekly): A-1 and A-2 are two representative grafts from the anti-CD45RB only treated group, while B-1 and B-2 from the anti-CD45RB plus anti-JE treated group

### DETAILED DESCRIPTION OF THE INVENTION

### Abbreviations

Abs antibodies, polyclonal or monoclonal; Ig immunoglobulin; Mab monoclonal antibody; V variable domain of an antibody; C constant domain of an antibody; H heavy chain of an antibody; L light chain of an antibody;

### DEFINITIONS

The term “antibody” herein is used in the broadest sense. As used herein, an “antibody” includes whole antibodies and any antigen binding fragment or a single chain thereof. Thus, the antibody includes any protein or peptide containing molecule that comprises at least a portion of an immunoglobulin molecule, such as but not limited to at least one complementarity-determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody of the present invention. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. 1988 Science 242:423-426, and Huston et al. 1988 Proc. Natl. Acad. Sci. USA 85:5879-5883. Such single chain antibodies are also intended to be encompassed within the term “antigen-binding portion” of an antibody. Antibody fragments are obtained using conventional techniques known to those with skill in the art, and the fragments are screened for utility in the same manner as are intact antibodies.

By “CCR2” is meant human CCR2A (MCP-1RA, NP_{—}000638) and/or human CCR2B (MCP-1RB, NP_{—}000639) and to proteins having an amino acid sequence which is the same as that of a naturally occurring or endogenous corresponding mammalian CCR2 protein (e.g., recombinant proteins). CCR2A, isoform A, has distinct C-terminus and is 14 amino acids longer than CCR2B, isoform B, due to alternative splicing in the coding region that results in a frameshift and use of a downstream stop codon (Charo, et al. 1994. Proc. Natl. Acad. Sci. U.S.A. 91 (7): 2752-2756). CCR2, as defined herein, includes mature receptor protein, polymorphic or allelic variants, and isoforms of a mammalian CCR2 (e.g., produced by alternative splicing or other cellular processes), and modified or unmodified forms of the foregoing (e.g., glycosylated, unglycosylated). Such proteins can be recovered or isolated from a source which naturally produces mammalian CCR2, for example.

A “CCR2 antagonist” prevents the biological functions or bioactivity associated with CCR2A or CCR2B in cells that display CCR2A or CCR2B or other isoforms or variants as defined herein. Antagonists included within the scope of the present invention include antibodies, synthetic or native sequence peptides and small molecule antagonists, which bind MCP-1/CCL2 or CCR2 or which prevent the binding of CCR2 with its cognate ligand(s) and thereby inhibit CCR2 biological functions. Thus, an inhibitor refers to substances including antagonists which bind receptor (e.g., an antibody, a mutant of a natural ligand, small molecular weight organic molecules, other competitive inhibitors of ligand binding), and substances which inhibit receptor function without binding thereto (e.g., an anti-idiotypic antibody).

The term “graft” as used herein refers to biological material derived from a donor for transplantation into a recipient. Grafts include such diverse material as, for example, isolated cells such as stem cells (embryonic or peripheral), islet cells; organized cellular structures and tissues such as pancreatic islets, the amniotic membrane of a newborn, bone marrow, hematopoietic precursor cells, and ocular tissue, such as corneal tissue, invertebral disc or cartilage; and organs such as skin, heart, liver, spleen, pancreas, thyroid lobe, lung, kidney, tubular organs (e.g., intestine, blood vessels, or esophagus), etc. The tubular organs can be used to replace damaged portions of esophagus, blood vessels, or bile duct. The skin grafts can be used not only for burns, but also as a dressing to damaged intestine or to close certain defects such as diaphragmatic hernia. The graft is derived from any mammalian source, including human, whether from cadavers or living donors. Preferably when the graft is allogeneic, the donor of the graft and the host are matched for HLA class II antigens.

By “MCP-1” is meant the 76 amino acid sequence referenced in NCBI record accession No. NP_{—}002973 and variously known as MCP (monocyte chemotactic protein), SMC-CF (smooth muscle cell chemotactic factor), LDCF (lymphocyte-derived chemotactic factor), GDCF (glioma-derived monocyte chemotactic factor), TDCF (tumor-derived chemotactic factors), HCl 1 (human cytokine 11), MCAF (monocyte chemotactic and activating factor). The gene symbol is SCYA2, the JE gene on human chromosome 17, and the new designation is CCL2 (Zlotnik, Yoshie 2000. Immunity 12:121-127). JE is the mouse homolog of human MCP-1/CCL2.

The term “transplant” and variations thereof refers to the insertion of a graft into a host, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from at least one other human. In another scenario, the graft is derived from a species different from that into which it is transplanted, such as a baboon heart transplanted into a human recipient host, and including animals from phylogenically widely separated species, for example, a pig heart valve, or animal beta islet cells or neuronal cells transplanted into a human host.

By “preventing chronic rejection” is meant that a remedy prevents, controls, slows, or reverses the occurrence of functional or histological signs of chronic rejection, when initiated before chronic rejection has resulted in graft failure either by long term or short term administration. A treatment capable of controlling chronic rejection is a treatment that slows the progression of the disease process, when initiated after functional or histological signs of chronic rejection are observed. Further, a treatment capable of reversing chronic rejection is a treatment that, when initiated after functional or histological signs of chronic rejection have appeared, reverses the disease process and returns functional and histological findings closer to normal. Therefore, “preventing chronic rejection” used in the present invention means protection or maintenance of transplanted organ or tissue for a long term. As used herein “treating” used in this invention means both treatments that comprise “controlling” and “reversing” the functional or histological signs of chronic rejection.

Mammals which maybe treated in the present invention include livestock mammals such as cows, houses, etc., domestic animals such as dogs, cats, rats, etc. and humans, preferably humans.

### CITATIONS

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2004); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, NY, N.Y., (1997-2004).

### Allograft Survival

Arterial patency and health is essential to the efficient functioning and regenerative capacity of all organs. The typical manifestation of chronic organ rejection is an arteriosclerosis-like alteration, such as transplant vasculopathy, graft vessel disease, graft arteriosclerosis, transplant coronary disease, angiostenosis, or interstitial fibrosis. This vascular lesion is characterized by migration and proliferation of smooth muscle cells, leading to intimal proliferation and thickening, smooth muscle cell hypertrophy repair, and finally to gradual luminal obliteration (vascular remodeling). Therefore, deterioration of the vascular system of a grafted organ or tissue may be clearly understood as the cause of graft failure.

Due to its enormous metabolic rate, cardiac tissue is profoundly affected by any reduction in perfusion capacity. Prevention, amelioration, or reversal of pathology related to vasculopathy is of critical importance in preserving cardiac grafts. Coronary artery disease is a late pathologic process common to all cardiac allografts. The pathology is characterized by myointimal hyperplasia of the small- and medium-sized vessels. The lesions are diffuse in nature. The lesions may appear any time from 3 months to several years after implantation. Currently, the process has no treatment other than retransplantation.

Early and persistent expression of MCP-1 in allografts has been implicated in the pathogenesis of transplant arteriosclerosis. MCP-1 secreted by leukocytes, endothelial cells, smooth muscle cells and infiltrating leukocytes in the grafts can result in tissue damage and tissue remodeling of the grafts. MCP-1 levels are elevated in renal allograft recipients experiencing chronic rejection (Boratynska, 1998 Pol Arch Med Wewn 99:272) and in animal models of chronic renal rejection (Nadeau, 1995 Proc Natl Acad Sci USA, 92: 8729). In a mouse model of obliterative bronchiolitis, a manifestation of chronic lung rejection, CCR2 knock-out or anti-JE (mouse homolog of MCP-1) mAb reduced the degree of tissue remodeling (Belperio, 2001 J Clin Investig 108:547-56). The presence of persistent MCP-1 positive macrophages in rat cardiac allografts has been previously noted (Russell, 1993. Proc Nat Acad Sci USA 90:6086-90). Blocking MCP-1 with a monoclonal antibody produced long-term islet allograft survival when combined with the anti-T cell drug, rapamycin (Lee et al. 2003 J Immunol 171:6929-35).

Based on applicants hypothesis that macrophage and monocyte/macrophage chemotactic factor MCP-1/CCL2 plays a key role in the development of arteriosclerosis and fibrosis associated with chronic organ rejection, the impact of an anti-JE (a mouse homolog of human MCP-1) monoclonal antibody (mAb) on chronic rejection of heterotopic cardiac allograft transplant was tested. It was discovered that anti-JE treatment is beneficial in preventing and controlling chronic rejection in terms of perivascular cellular infiltration, arteritis, and fibrosis. Most importantly, it was discovered that anti-JE significantly suppressed arterial intimal proliferation/thickening, which is the hallmark of renal and cardiac chronic rejection. These data provide support of the use of anti-MCP-1 antibody for the prevention and control of chronic rejection in allograft transplantation.

### Compounds of the Invention

MCP-1 is known to bind and signal through the chemokine receptor CCR2. CCR2 is a seven trans-membrane-spanning G-protein-coupled receptor expressed on many cells including monocytes, T-cells, B-cells, and basophils. Two MCP-1 specific receptors, CCR2A and CCR2B, have been cloned which signal in response to nanomolar (nM) concentrations of MCP-1. CCR2A (CC-CKR2A) and CCR2B (CC-CKR2A) represent two cDNAs that encode two MCP-1-specific receptors with alternatively spliced carboxyl tails. MCP-1 binds to both isoforms with high affinity MCP-1 induces calcium flux in cells expressing CCR2B but not in cells expressing CCR2A. 5-fold less MCP-1 induces chemotaxis in cells expressing CCR2B compared to cells expressing CCR2A.

Other proteins with certain functional and sequence homology to human MCP-1 are known. Especially similar to MCP-1 (GenBank NP_{—}002973) are MCP-2 (GenBank NP_{—}005614) and eotaxin (GenBank P-51671); MCP-2 having 61.8 percent and eotaxin-1 having 63.2 percent sequence identity to MCP-1. The range of activities and spectrum of involvement of these proteins in human homeostatic mechanisms and pathology is not as well understood for the homologs of MCP-1. For example, MCP-2 (renamed CCL8) is related closely to MCP-1 and MCP-3 (renamed CCL7, Genbank NP_{—}006264) and uses both CCR1 as well as CCR2B as its functional receptors. MCP-3 binds to a receptor designated D6. MCP-3 also binds to CCR10 and CCR1. The MCP-3 protein (97 amino acids) sequence shows 74 percent identity with MCP-1 and 58 percent homology with MCP-2. Secreted MCP-3 differs from MCP-1 in being N-glycosylated. MCP-4 (renamed CCL13, Genbank NP_{—}005399) shares 56-61 percent sequence identity with the three known monocyte chemotactic proteins and is 60 percent identical with Eotaxin-1. The functions of MCP-4 appear to be highly similar to those of MCP-3 and Eotaxin. Like MCP-3, MCP-4 is a potent chemoattractant for monocytes and T-lymphocytes. It is inactive on neutrophils. On monocytes, MCP-4 binds to receptors that recognize MCP-1, MCP-3, RANTES (CCL5), and eotaxin, the CCR1 and CCR3 receptors, and shows full cross-desensitization with eotaxin-1. MCP-5 is murine CC-chemokine and related most closely to human MCP-1 (66% amino acid identity). The gene symbol for MCP-5 is SCYA12 (renamed CCL12). Cells transfected with the chemokine receptor CCR2 have been shown to respond to MCP-5. For general information on cytokines and chemokines see http://www.copewithcytokines.de/cope.cgi and for the current classification system, Zlotnik A., Yoshie O. 2000. Chemokines: a new classification system and their role in immunity. Immunity 12:121-127.

The forgoing discussion serves to emphasize that an antagonist may prevent the biological function of CCR2 binding by either direct action on CCR2 or one of its ligands, CCL2, CCL7, CCL8. In one aspect of the invention, the antagonist binds to MCP-1/CCL2 and neutralizes its ability to bind to CCR2.

Anti-CCR2 antibodies are disclosed in U.S. Pat. No. 6,084,075, U.S. Pat. No. 6,458,353 and U.S. Pat. No. 6,696,550. In one embodiment of the method of the invention, a method of inhibiting the biological interaction of a cell bearing mammalian CCR2 with a chemokine, comprises contacting said cell with an effective amount of an antibody or functional fragment thereof which binds to CCR2 or a portion of said receptor. In one embodiment, the antibody is monoclonal antibody (mAb) LS132.1D9 (1D9) or an antibody, which can compete with 1D9 for binding to human CCR2 or a portion of human CCR2. Functional fragments of the foregoing antibodies are also envisioned.

Antibodies capable of binding MCP-1 have been reported: JP9067399 discloses an antibody obtained from isolated blood cells and JP05276986 discloses a hybridoma secreting an IgM anti-human MCP-1. More recently, antibodies capable of binding a plurality of beta-chemokines including MCP-1 were disclosed (WO03048083) and an MCP-1 binding antibody which also binds eotaxin (US20040047860). Antibodies which selectively bind and neutralize mouse homologs of human MCP-1/CCL2 or human MCP-1/CCL2 are disclosed in applications co-pending patent applications U.S. Ser. No. 11/170,453 and 60/682,654.

In one embodiment of the invention, the CCR2 antagonist is the anti-human MCP-1/CCL2 antibody designated C775 which can be produced by a cell line designated C1142 as disclosed in applications co-pending patent applications U.S. Ser. No. 11/170,453, variants such as humanized or reshaped forms, truncated forms, or binding fragments thereof as defined herein. In another embodiment, the CCR2 antagonist is the anti-human MCP-1/CCL2 antibody designated CNT0888 as disclosed in applications co-pending patent applications 60/682,654, variants, truncated forms, or binding fragments thereof as defined herein.

MCP-1/CCL2 truncations, variants, mutant proteins or “muteins” having the ability to bind CCR2 and have antagonistic activity may also be used to practice the method of the invention. Variants of homodimer-forming chemokines, such as CCL2, having a single amino acid substitution in the dimerization interface that alters the pattern of hydrogen bonds, so as to result in an obligate monomer that binds to the receptor and has agonistic properties in vitro but which can antagonize natural chemokines and have anti-inflammatory activity in vivo as taught in WO05037305A1 are among the variants useful in practicing the present invention. A peptide antagonist of MCP 1, is the truncated MCP-1 (9-76), which was shown both to prevent disease onset and to reduce disease symptoms in a mouse model of arthritis (Jiang-Hong Gong, et al., J. Exp. Med. 1997, 186:131).

### Modulation of CCR2/CCL2 Expression

An alternate method of antagonizing the interaction of CCR2 with its ligands, is by knocking down the expression of the CCR2 or its ligands, especially MCP-1/CCL2, using e.g. methods of RNA silencing. Thus, in another embodiment, compounds useful in practicing the method of the invention are nucleic acids, including oligonucleotides and polynucleotides in sense or antisense orientation, and single or double stranded nucleic acid molecules (e.g., siRNA) that target MCP-1 sequences and interfere with MCP-1 gene expression or that target CCR2 and interfere with CCR2 gene expression.

Gene expression can be modulated in several different ways, including by the use of siRNAs, shRNAs, antisense molecules and DNAzymes. SiRNAs and shRNAs both work via the RNAi pathway and have been successfully used to suppress the expression of genes. RNAi was first discovered in worms and the phenomenon of gene silencing related to dsRNA was first reported in plants by Fire and Mello (Fire et al., 1998. Nature 391: 806) and is thought to be a way for plant cells to combat infection with RNA viruses. In this pathway, the long dsRNA viral product is processed into smaller fragments of 21-25 bp in length by a DICER-like enzyme and then the double-stranded molecule is unwound and loaded into the RNA induced silencing complex (RISC). A similar pathway has been identified in mammalian cells with the notable difference that the dsRNA molecules must be smaller than 30 bp in length in order to avoid the induction of the so-called interferon response, which is not gene specific and leads to the global shut down of protein synthesis in the cell.

Synthetic siRNAs can be designed to specifically target one gene and they can easily be delivered to cells in vitro or in vivo. ShRNAs are the DNA equivalents of siRNA molecules and have the advantage of being incorporated into the cells' genome and then being replicated during every mitotic cycle.

DNAzymes have also been used to modulate gene expression. DNAzymes are catalytic DNA molecules that cleave single-stranded RNA. They are highly selective for the target RNA sequence and as such can be used to down-regulate specific genes through targeting of the messenger RNA.

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

siRNAs are double stranded RNAs that include the target sequence and its complement. Two uridine residues are added to the 3′ end of the RNAs (Elbashir et al. 2001 Nature 411:494-498).

RNA interference (RNAi) is now being used routinely in mammalian cells to study the functional consequences of reducing the expression of specific genes. RNAi is induced by transfecting small interfering RNAs (siRNAs), comprising double-stranded RNA molecules ˜21 nt in length with 2 nt 3′ overhangs (Elbashir et al. 2001 supra), or hairpin-forming 45-50mer (shRNA) molecules (Paddison, P J, et al., 2002. Genes & Development 16:948-958), that are complementary to the gene of interest. When transfected into mammalian cells, siRNA expression plasmids and have been shown to reduce the levels of both exogenous and endogenous gene products. Although they require more effort to prepare than chemically synthesized or in vitro transcribed siRNAs, the siRNA vectors can provide longer term reduction in target gene expression when coexpressed with a selectable marker (Brummelkamp, T R, et al., 2002. Science 296:550-553).

### Non-Protein, Non-Oligonucleic Acid Antagonists

Small molecule drugs and peptidomimetics can also be antagonists of CCR2. For example, WO04069809, WO04069810, WO05118574, WO06015986 teach mercaptoimidazoles as CCR2 receptor antagonists. Other small molecules exhibiting the desired biological properties can be selected by screening using methods such as those described herein and will have the property of preventing chronic rejection and prolonging graft survival.

### Methods of Making Antibodies

CCR2 antagonist antibodies of the present invention can be optionally produced by a variety of techniques, including the standard somatic cell hybridization technique (hybridoma method) of Kohler and Milstein (1975) Nature 256:495. In the hybridoma method, a mouse or other appropriate host animal, such as a hamster or macaque monkey, is immunized as described herein to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The CCR2 antagonistic antibody can also be optionally generated by immunization of a transgenic animal (e.g., mouse, rat, hamster, non-human primate, and the like) capable of producing a repertoire of human antibodies, as described herein and/or as known in the art. Cells that produce, e.g. a human anti-MCP-1 antibody can be isolated from such animals and immortalized using suitable methods, such as the methods described herein.

The use of transgenic mice carrying human immunoglobulin (Ig) loci in their germline configuration provide for the isolation of high affinity fully human monoclonal antibodies directed against a variety of targets including human self antigens for which the normal human immune system is tolerant (Lonberg, N. et al., U.S. Pat. No. 5,569,825, U.S. Pat. No. 6,300,129 and 1994, Nature 368:856-9; Green, L. et al., 1994, Nature Genet. 7:13-21; Green, L. & Jakobovits, 1998, Exp. Med. 188:483-95; Lonberg, N. and Huszar, D., 1995, Int. Rev. Immunol. 13:65-93; Kucherlapati, et al. U.S. Pat. No. 6,713,610; Bruggemann, M. et al., 1991, Eur. J. Immunol. 21:1323-1326; Fishwild, D. et al., 1996, Nat. Biotechnol. 14:845-851; Mendez, M. et al., 1997, Nat. Genet. 15:146-156; Green, L., 1999, J. Immunol. Methods 231:11-23; Yang, X. et al., 1999, Cancer Res. 59:1236-1243; Brüggemann, M. and Taussig, M J., Curr. Opin. Biotechnol. 8:455-458, 1997; Tomizuka et al. WO02043478). The endogenous immunoglobulin loci in such mice can be disrupted or deleted to eliminate the capacity of the animal to produce antibodies encoded by endogenous genes. In addition, companies such as Abgenix, Inc. (Freemont, Calif.) and Medarex (San Jose, Calif.) can be engaged to provide human antibodies directed against a selected antigen using technology as described above.

Preparation of Immunogenic Antigens, and Monoclonal Antibody production can be performed using any suitable technique such as recombinant protein production. The immunogenic antigens can be administered to an animal in the form of purified protein, or protein mixtures including whole cells or cell or tissue extracts, or the antigen can be formed de novo in the animal's body from nucleic acids encoding said antigen or a portion thereof.

Immunization with antigen can be optionally accompanied by addition of an adjuvant, such as complete Freund's adjuvant. The immune response can be monitored over the course of the immunization protocol with plasma samples being obtained by retroorbital bleeds. The plasma can be screened by ELISA (as described below), and mice with sufficient titers of anti-MCP-1 immunoglobulin can be used for fusions. Mice can be boosted intravenously with antigen 3 days before sacrifice and removal of the spleen. It is expected that 2-3 fusions for each antigen may need to be performed. Several mice will be immunized for each antigen.

To generate hybridomas producing monoclonal CCR2 antagonist antibodies, splenocytes and lymph node cells from immunized mice can be isolated and fused to an appropriate immortalized cell line, such as a mouse myeloma cell line. The resulting hybridomas can be screened for the production of antigen-specific antibodies.

A suitable immortal cell line incapable of producing immunoglobulin chains is selected as a fusion partner, e.g., a myeloma cell line such as, but not limited to, Sp2/0 and derivative cell lines, NS1 and derivatives, especially NSO engineered NSO lines such as GS-NSO, AE-1, L.5, P3X63Ag8.653, U937, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O or the like, or heteromyelomas, fusion products thereof, or any cell or fusion cell derived therefrom, or any other suitable cell line as known in the art (Birch et al. 1994. Biologics 22:127-133). The fused cells (hybridomas) or recombinant cells can be isolated using selective culture conditions or other suitable known methods, and cloned by limiting dilution or cell sorting, or other known methods. Cells which produce antibodies with the desired specificity can be detected by a suitable assay (e.g., ELISA) and selected for manipulation.

Other suitable methods of generating or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Bioinvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590689 (Ixsys, now Applied Molecular Evolution (AME), each entirely incorporated herein by reference) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Screening antibodies for specific binding to similar proteins or fragments can also be conveniently achieved using peptide display libraries. This method involves the screening of large collections of peptides for individual members having the desired function or structure. Antibody screening using peptide display libraries is well known in the art. The displayed peptide sequences can be from 3 to 5000 or more amino acids in length, frequently from 5-100 amino acids long, and often from about 8 to 25 amino acids long. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; 5,223,409, 5,403,484, 5,571,698, 5,837,500, assigned to Dyax, 5,427,908, 5,580,717, assigned to Affymax; 5,885,793, assigned to Cambridge antibody Technologies; 5,750,373, assigned to Genentech, 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

### Antibody Fragments

Antibody fragments can be derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. F(ab′)2, Fab, Fv and ScFv antibody fragments can all be expressed in and secreted from mammalian host cells or from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)).

In other embodiments, the antibody of is a single chain Fv fragment (scFv). See WO 93/16185; U.S. Pat. No. 5,571,894; and U.S. Pat. No. 5,587,458. Fv and sFv are species with intact combining sites, that is a VH and VL domain, that are devoid of constant regions. Typically, the VH and VL domains are cloned and re-engineered to lie within a single polypeptide and connected by a flexible linker long enough to allow interaction of the two domains within the single polypeptide. Alternatively, fusion proteins may be constructed to yield fusion of an effector protein at either the amino or the carboxy terminus of an sFv. See Antibody Engineering, 1995. ed. Borrebaeck.

### Methods of Identifying Antagonists

Antagonists of CCR2 biological activity can be identified using suitable in vitro assays and in vivo models as exemplified hereinbelow.

Binding inhibition assays can be used to identify antibodies or fragments thereof which bind CCR2 and inhibit binding of another compound such as a ligand (e.g., MCP-1, MCP-2, MCP-3 and/or MCP-4) to CCR2 or a functional variant. For example, a binding assay can be conducted in which a reduction in the binding of a ligand of CCR2 (in the presence of an antibody), as compared to binding of the ligand in the absence of the antibody, is detected or measured. A composition comprising an isolated and/or recombinant mammalian CCR2 or functional variant thereof can be contacted with the ligand and antibody simultaneously, or one after the other, in either order. A reduction in the extent of binding of the ligand in the presence of the antibody, is indicative of inhibition of binding by the antibody. For example, binding of the ligand could be decreased or abolished.

In one embodiment, direct inhibition of the binding of a ligand (e.g., a chemokine such as MCP-1/CCL2) to a mammalian CCR2 or variant thereof by an antibody or fragment is monitored. For example, the ability of an antibody to inhibit the binding of ¹²⁵I-labeled MCP-1, ¹²⁵I-labeled MCP-2, ¹²⁵I-labeled MCP-3 or ¹²⁵I-labeled MCP-4 to mammalian CCR2 can be monitored. Such an assay can be conducted using suitable cells bearing CCR2 or a functional variant thereof, such as isolated blood cells (e.g., T cells, PBMC) or a suitable cell line naturally expressing CCR2, or a cell line containing nucleic acid encoding a mammalian CCR2, or a membrane fraction from said cells, for instance.

Other methods of identifying the presence of an antibody which binds CCR2 are available, such as other suitable binding assays, or methods which monitor events which are triggered by receptor binding, including signaling function and/or stimulation of a cellular response (e.g., leukocyte trafficking).

It will be understood that the inhibitory effect of antibodies of the present invention can be assessed in a binding inhibition assay. Competition between antibodies for receptor binding can also be assessed in the method. Antibodies which are identified in this manner can be further assessed to determine whether, subsequent to binding, they act to inhibit other functions of CCR2 and/or to assess their therapeutic utility.

### Signaling Assays

The binding of a ligand or promoter, such as an agonist, to CCR2 can result in signaling by this G protein-coupled receptor, and the activity of G proteins as well as other intracellular signaling molecules is stimulated. The induction of signaling function by a compound (e.g., an antibody or fragment thereof) can be monitored using any suitable method. Such an assay can be used to identify antibody agonists of CCR2. The inhibitory activity of an antibody or functional fragment thereof or other CCR2 antagonist compound candidate can be determined using a ligand or promoter in the assay, and assessing the ability of the antibody to inhibit the activity induced by ligand or promoter.

G protein activity, such as hydrolysis of GTP to GDP, or later signaling events triggered by receptor binding, such as induction of rapid and transient increase in the concentration of intracellular (cytosolic) free calcium [Ca2+]I, can be assayed by methods known in the art or other suitable methods (see e.g., Neote, K. et al., Cell, 72: 415-425 1993); Van Riper et al., J. Exp. Med., 177: 851-856 (1993); Dahinden, C. A. et al., J. Exp. Med., 179: 751-756 (1994)).

For example, the functional assay of Sledziewski et al. using hybrid G protein coupled receptors can be used to monitor the ability a ligand or promoter to bind receptor and activate a G protein (Sledziewski et al., U.S. Pat. No. 5,284,746, the teachings of which are incorporated herein by reference).

Such assays can be performed in the presence of the antibody or fragment thereof to be assessed, and the ability of the antibody or fragment to inhibit the activity induced by the ligand or promoter is determined using known methods and/or methods described herein.

### Chemotaxis and Assays of Cellular Stimulation

Chemotaxis assays can also be used to assess the ability of an antibody or functional fragment thereof ody agonists of CCR2. The inhibitory activity of an antibody or functional fragment thereof or other CCR2 antagonist compound candidate to block binding of a ligand to mammalian CCR2 or functional variant thereof and/or inhibit function associated with binding of the ligand to the receptor. These assays are based on the functional migration of cells in vitro or in vivo induced by a compound. Chemotaxis can be assessed, e.g., in an assay utilizing a 96-well chemotaxis plate, or using other art-recognized methods for assessing chemotaxis. For example, the use of an in vitro transendothelial chemotaxis assay is described by Springer et al. (Springer et al., WO 94/20142, published Sep. 15, 1994, the teachings of which are incorporated herein by reference; see also Berman et al., Immunol. Invest. 17: 625-677 (1988)). Migration across endothelium into collagen gels has also been described (Kavanaugh et al., J. Immunol., 146: 4149-4156 (1991)). Stable transfectants of mouse L1-2 pre-B cells or of other suitable host cells capable of chemotaxis can be used in chemotaxis assays, for example.

Generally, chemotaxis assays monitor the directional movement or migration of a suitable cell (such as a leukocyte (e.g., lymphocyte, eosinophil, basophil)) into or through a barrier (e.g., endothelium, a filter), toward increased levels of a compound, from a first surface of the barrier toward an opposite second surface. Membranes or filters provide convenient barriers, such that the directional movement or migration of a suitable cell into or through a filter, toward increased levels of a compound, from a first surface of the filter toward an opposite second surface of the filter, is monitored. In some assays, the membrane is coated with a substance to facilitate adhesion, such as ICAM-1, fibronectin or collagen. Such assays provide an in vitro approximation of leukocyte “homing”.

For example, one can detect or measure inhibition of the migration of cells in a suitable container (a containing means), from a first chamber into or through a microporous membrane into a second chamber which contains an antibody to be tested, and which is divided from the first chamber by the membrane. A suitable membrane, having a suitable pore size for monitoring specific migration in response to compound, including, for example, nitrocellulose, polycarbonate, is selected. For example, pore sizes of about 3-8 microns, and preferably about 5-8 microns can be used. Pore size can be uniform on a filter or within a range of suitable pore sizes.

To assess migration and inhibition of migration, the distance of migration into the filter, the number of cells crossing the filter that remain adherent to the second surface of the filter, and/or the number of cells that accumulate in the second chamber can be determined using standard techniques (e.g., microscopy). In one embodiment, the cells are labeled with a detectable label (e.g., radioisotope, fluorescent label, antigen or epitope label), and migration can be assessed in the presence and absence of the antibody or fragment by determining the presence of the label adherent to the membrane and/or present in the second chamber using an appropriate method (e.g., by detecting radioactivity, fluorescence, immunoassay). The extent of migration induced by an antibody agonist can be determined relative to a suitable control (e.g., compared to background migration determined in the absence of the antibody, compared to the extent of migration induced by a second compound (i.e., a standard), compared with migration of untransfected cells induced by the antibody).

In one embodiment, particularly for T cells, monocytes or cells expressing a mammalian CCR2, transendothelial migration can be monitored. In this embodiment, transmigration through an endothelial cell layer is assessed. To prepare the cell layer, endothelial cells can be cultured on a microporous filter or membrane, optionally coated with a substance such as collagen, fibronectin, or other extracellular matrix proteins, to facilitate the attachment of endothelial cells. Preferably, endothelial cells are cultured until a confluent monolayer is formed. A variety of mammalian endothelial cells can are available for monolayer formation, including for example, vein, artery or microvascular endothelium, such as human umbilical vein endothelial cells (Clonetics Corp, San Diego, Calif.). To assay chemotaxis in response to a particular mammalian receptor, endothelial cells of the same mammal are preferred; however endothelial cells from a heterologous mammalian species or genus can also be used.

Generally, the assay is performed by detecting the directional migration of cells into or through a membrane or filter, in a direction toward increased levels of a compound, from a first surface of the filter toward an opposite second surface of the filter, wherein the filter contains an endothelial cell layer on a first surface. Directional migration occurs from the area adjacent to the first surface, into or through the membrane, towards a compound situated on the opposite side of the filter. The concentration of compound present in the area adjacent to the second surface, is greater than that in the area adjacent to the first surface.

In one embodiment used to test for an antibody inhibitor, a composition comprising cells capable of migration and expressing a mammalian CCR2 receptor can be placed in the first chamber. A composition comprising one or more ligands or promoters capable of inducing chemotaxis of the cells in the first chamber (having chemoattractant function) is placed in the second chamber. Preferably shortly before the cells are placed in the first chamber, or simultaneously with the cells, a composition comprising the antibody to be tested is placed, preferably, in the first chamber. Antibodies or functional fragments thereof which can bind receptor and inhibit the induction of chemotaxis, by a ligand or promoter, of the cells expressing a mammalian CCR2 in this assay are inhibitors of receptor function (e.g., inhibitors of stimulatory function). A reduction in the extent of migration induced by the ligand or promoter in the presence of the antibody or fragment is indicative of inhibitory activity. Separate binding studies (see above) could be performed to determine whether inhibition is a result of binding of the antibody to receptor or occurs via a different mechanism.

In vivo assays which monitor leukocyte infiltration of a tissue, in response to injection of a compound (e.g., chemokine or antibody) in the tissue, are described below (see Models of Inflammation). These models of in vivo homing measure the ability of cells to respond to a ligand or promoter by emigration and chemotaxis to a site of inflammation and to assess the ability of an antibody or fragment thereof to block this emigration.

In addition to the methods described, the effects of an antibody or fragment on the stimulatory function of CCR2 can be assessed by monitoring cellular responses induced by active receptor, using suitable host cells containing receptor.

### Identification of Additional Ligands and Inhibitors of Mammalian CCR2 Function

The assays described above, which can be used to assess binding and function of the antibodies and fragments of the present invention, can be adapted to identify additional ligands or other substances which bind a mammalian CCR2 or functional variant thereof, as well as inhibitors and/or promoters of mammalian CCR2 function. For example, agents having the same or a similar binding specificity as that of an antibody of the present invention or functional portion thereof can be identified by a competition assay with said antibody or portion thereof. Thus, the present invention also encompasses methods of identifying ligands of the receptor or other substances which bind a mammalian CCR2 protein, as well as inhibitors (e.g., antagonists) or promoters (e.g., agonists) of receptor function. In one embodiment, cells bearing a mammalian CCR2 protein or functional variant thereof (e.g., leukocytes, cell lines or suitable host cells which have been engineered to express a mammalian CCR2 protein or functional variant encoded by a nucleic acid introduced into said cells) are used in an assay to identify and assess the efficacy of ligands or other substances which bind receptor, including inhibitors or promoters of receptor function. Such cells are also useful in assessing the function of the expressed receptor protein or polypeptide.

According to the present invention, ligands and other substances which bind receptor, inhibitors and promoters of receptor function can be identified in a suitable assay, and further assessed for therapeutic effect. Inhibitors of receptor function can be used to inhibit (reduce or prevent) receptor activity, and ligands and/or promoters can be used to induce (trigger or enhance) normal receptor function where indicated. Thus, the present invention provides a method of treating graft rejection, comprising administering an inhibitor of receptor function to an individual (e.g., a mammal).

### Pharmaceutical Compositions Comprising CCR2 Antagonists

The invention includes methods for preparing pharmaceutical compositions for modulating the transcription, expression, or activity of a CCR2. Such methods comprise formulating a pharmaceutically acceptable carrier with an agent that modulates expression or activity of a CCR2. Such compositions can further include additional active agents. Thus, the invention further includes methods for preparing a pharmaceutical composition by formulating a pharmaceutically acceptable carrier with an agent that modulates expression or activity of a CCR2 and one or more additional active compounds.

Pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, histadine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), PLURONICS® and hyaluronic acid (HA).

Formulations may be designed to optimize stability of the CCR2 antagonist or, additionally, allow for sustained or extended release of the active into the bloodstream. Suitable formulations for each of type of CCR2 antagonist and route of administration may be found in, for example, “Remington: The Science and Practice of Pharmacy”, A. Gennaro, ed., 20th edition, Lippincott, Williams & Wilkins, Philadelphia, Pa., 2000.

In order for the formulations to be used for in vivo administration, they must be sterile. The formulation may be rendered sterile by filtration through sterile filtration membranes, prior to or following lyophilization and reconstitution. The therapeutic compositions herein generally are placed into a container having a sterile access port, for example, an intravenous solution bag or vial having a stopper pierceable by a hypodermic injection needle. Therapeutic compositions can be administered with medical devices known in the art.

### Methods of Treatment

### Patient Assessment

Assessment of a transplant patient (graft recipient) for the need of anti-CCR2 therapy can be performed at any time prior to, concurrent with, or subsequent to the transplant (graft transfer) procedure itself using methods known to those skilled in the art. Generally, methods used to monitor the hallmarks of chronic rejection, vascular pathologies, which can be prevented, ameliorated, or reversed by treatment with anti-CCR2 therapy are useful in assessing the need of a patient for therapy. Methods in use, as outlined herein below, and those yet to be developed may all be employed in patient assessment and evaluation of the need for anti-CCR2 therapy.

In the case of cardiac transplantation, the postoperative patient is maintained on a combination of pressor agents while the donor heart regains energy stores. The ionized calcium level of the patient is carefully monitored and replaced with calcium chloride because the function of the denervated heart is initially extremely dependent on circulating calcium ions. The acid-base status of the patient is also carefully monitored and corrected. Once stabilized, the patient is rapidly weaned from the ventilator and the pressors. The posttransplant hospital stay can be as short as 5 days, depending upon the condition of the recipient prior to surgery.

Immunosuppression is started soon after surgery. Several regimens can be used and is dependent upon the training and experience of the transplant center. Most cardiac transplant programs currently use a three-drug regimen including a calcineurin inhibitor (cyclosporine or tacrolimus), an inhibitor of T cell proliferation or differentiation (azathioprine, mycophenolate mofetil, or sirolimus), and at least a short initial course of glucocorticoids. Many programs also include an initial “induction” course of polyclonal or monoclonal anti-T cell antibodies in the perioperative period to decrease the frequency or severity of early posttransplant rejection. Monoclonal antibodies (daclizumab and basiliximab), which block the interleukin-2 receptor and may provide prevention of allograft rejection without additional global immunosuppression and specific antilymphocyte therapy, e.g. anti-CD20 Mab, may also be used.

Diagnosis of cardiac allograft rejection is usually made with the use of endomyocardial biopsy, either on a surveillance basis or in response to clinical deterioration. Therapy for acute rejection consists of augmentation of immunosuppression, the intensity and duration of which is dictated by the severity of the rejection. The frequency of visits gradually diminishes until the patient is generally seen on an annual basis. Cardiac allograft recipients are prone to develop coronary artery disease (CAD), which is generally a diffuse, concentric, and longitudinal process and which is notably different from “ordinary” atherosclerotic CAD, which is more focal and often eccentric. Certain centers perform coronary angiography annually after transplantation to monitor the patient for the development of allograft vascular disease.

Retransplantation is the only definitive form of therapy for advanced allograft CAD, however, retransplantation procedures have inferior survival rates.

In renal transplant patients, while 1-year transplant survival is excellent, most recipients experience progressive decline in kidney function over time thereafter. The chronic renal transplant dysfunction can be caused by recurrent disease, hypertension, cyclosporine or tacrolimus nephrotoxicity, chronic immunologic rejection, secondary focal glomerulosclerosis, or a combination of these pathophysiologies. Chronic vascular changes with intimal proliferation and medical hypertrophy are commonly found. Control of systemic and intrarenal hypertension with ACE inhibitors is thought to have a beneficial influence on the rate of progression of chronic renal transplant dysfunction. Renal biopsy can distinguish subacute cellular rejection from recurrent disease or secondary focal sclerosis.

Hypertension may be caused by (1) native kidneys; (2) rejection activity in the transplant; (3) renal artery stenosis, if an end-to-end anastomosis was constructed with an iliac artery branch; and (4) renal calcineurin inhibitor toxicity. Recipients of renal transplants have a high prevalence of coronary artery and peripheral vascular diseases. The percentage of deaths from these causes has been slowly rising as the numbers of transplanted diabetic patients and the average age of all recipients increase. More than 50% of renal recipient mortality is attributable to cardiovascular disease. In addition to strict control of blood pressure and blood lipid levels, close monitoring of patients for indications of further medical or surgical intervention is an important part of management.

In liver transplant patients, hepatic artery, celiac trunk, superior mesenteric artery, portal vein, superior mesenteric vein, splenic vein, hepatic veins, and inferior vena cava (IVC) may all be sites for thrombosis or stenosis. Hepatic artery stenosis (HAS) and hepatic artery thrombosis (HAT) usually requires operative vascular reconstruction or retransplantation although balloon angioplasty is sometime successful. Chronic rejection is a relatively rare outcome that can follow repeated bouts of acute rejection or that occurs unrelated to preceding rejection episodes. Morphologically, chronic rejection is characterized by progressive cholestasis, focal parenchymal necrosis, mononuclear infiltration, vascular lesions (intimal fibrosis, subintimal foam cells, fibrinoid necrosis), and fibrosis. This process may be reflected as ductopenia or the vanishing bile duct syndrome. Some of the histologic hallmarks of chronic rejection may be so similar to those of chronic viral hepatitis that differentiation between the two may be difficult. Reversibility of chronic rejection is limited; in patients with therapy-resistant chronic rejection, retransplantation is a possibility.

In most patients, direct Doppler evaluation of hepatic artery anastomosis is not possible because the donor-recipient arterial anastomosis is tortuous, because it is in an inconsistent position, and because it is usually obscured by overlying bowel gas. Because the specificity of Doppler is only 64% in detecting marked arterial disease (ie, HAT or hemodynamically significant HAS), angiography usually is required to confirm the diagnosis. Three-dimensional gadolinium-enhanced MR angiography may have the potential to enable accurate diagnosis of vascular complications of liver transplantation.

In patients who recipients of other types of grafted organs, tissues and cells, including lung and heart-lung transplant patients, islet and islet cell recipients, and hand and face transplant patients; graft health can be monitored by methods known to those specialized in and practicing in the field. It is anticipated by the applicants that the need to prevent chronic rejection and graft failure can be universally met by anti-CCR2 therapy.

In the methods of the invention, a CCR2 antagonist can be administered at the time of onset of detectable markers for chronic rejection as are known in the present are or those that may subsequently be found to be of value in monitoring graft health in transplant patients. Further, the therapeutic effect of reversing and/or preventing chronic rejection by the use of CCR2 antagonists can be monitored accordingly based on testing for said detectable markers in the transplant recipient in fluids such as blood or serum or biopsy samples, or through the use of monitoring devices and procedures such as Doppler ultrasound, magnetic resonance (MR), and contrast imaged angiography.

### Routes of Administration

The route of administration is in accordance with known and accepted methods, e.g., injection or infusion by intravenous, intraperitoneal, intramuscular, intraarterial, via the portal vein; topical administration, by sustained release or extended-release means; subcutaneous injection, by transmucosal or transdermal delivery, through topical applications, nasal spray, suppository and the like, or may be administered orally.

### Dosages

The dose of anti-CCR2 antagonist which appropriate to prevent, ameliorate, reverse, or halt the progression of chronic rejection in a patient in need thereof will be found empirically and will be dependent on the potency of the active agent, the strength of the formulation and the duration of the effective level of the agent following administration in the body of the recipient.

The course of treatment may be chronic or continuous administration in a continuous mode as opposed to an acute mode, so as to maintain the initial therapeutic effect (activity) for an extended period of time. Alternatively, the treatment may be intermittent or cyclic in nature in order to provide periods of acute antagonist activity followed by periods of lower or no antagonist activity in the body of the patient. Thus, the dosage schedule can be varied, such that the antibody is administered once, twice, three or more times per week for any number of weeks or the antibody is administered more than once (e.g., two, three, four, five, six, seven times) with administration occurring once a week, once every two, three, four, five, six, seven, eight, nine or ten weeks.

In the case of monoclonal antibody antagonist of CCR2 bioactivity, the agent will generally be administered at an amount which si based on the body weight of the recipient, e.g. between 0.1 and 100 mg/kg per course of therapy. An exemplary, non-limiting range for a therapeutically or prophylactically effective amount of an antibody administered according to the methods of the invention is 0.1-20 mg/kg, more preferably 1-10 mg/kg. In one embodiment, the anti-CCR2 or anti-CCL2 antibody can be administered by intravenous infusion at a rate of less than 10 mg/min, preferably less than or equal to 5 mg/min to reach a dose of about 1 to 500 mg/m2, preferably about 10 to 400 mg/m2, about 18 to 350 mg/m2, and more preferably, about 250-280 mg/m2. The anti-CCR2 or anti-CCL2 antibody can be administered in a single dose or in multiple doses.

### Combination Therapy

Transplantation pharmacology is among the most complex and long-term of any regimen developed for maintenance of a human patient. Allogeneic grafts will not survive transplantation to a new host unless the recipient immune system is downregulated or suppressed. Furthermore, downregulation through immunomodulation must be maintained on a lifelong basis because antigen (the allograft) in solid organ transplantation is always present and continually renewed. There are two main strategies for achieving this objective: immunosuppressive therapy and tolerance induction. Immunosuppressive therapy has been used in transplantation since the 1950s. Tolerance induction is currently under intensive investigation. Thus, it is anticipated that the methods of the invention using CCR2 antagonists will not be practiced as the sole means of graft maintenance or survival.

At the present time, maintenance immunosuppressive therapy relies on nonspecific immunosuppression with corticosteroids along with more specifically targeted therapy. A number of preparations of agents which interfere with discrete sites in the T- and B-cell activation cascades exist today, including cyclosporine formulations (CsA, SANDIMMUNE®, NEORAL®, and SANGCYA®) and tacrolimus (TAC, PROGRAF®) which target calcineurin and inhibit cytokine transcription, azathioprine (AZA, a pro form of 6-mercaptopurine) and mycopheno late mofetil (MMF, CELLCEPT®) or a delayed-release tablet form of mycophenolic acid (MYFORTIC®) which inhibit nucleotide synthesis, and sirolimus (SRL, RAPAMUNE®) and everolimus (ERL, CERTICAN®) which inhibit growth factor signal transduction.

Other immunosuppressive T cell-directed agents are antibodies such as the anti-IL2 receptor CD25 antibodies daclizumab (ZENAPAX®) and basiliximab (SIMULECT®) inhibit stimulation of T cell interleukin (IL)-2 receptor sites by IL-2, polyclonal anti-lymphocyte antibody, anti-CD3 antibody (muromomab, OKT3, ORTHOCLONE®), and LFA-1 antibody (RAPTIVA®). Two anti-thymocyte globulin preparations (ATG) preparations are currently available: THYMOGLOBULIN (rabbit ATG [rATG]) and ATGAM (equine ATG).

Despite the advantages these drugs offer with respect to their immunosuppressive, anti-inflammatory, and anti-parasitic activities, there are numerous adverse effects associated with these immunosuppressive agents. For example, cyclosporine A therapy adverse effects include nephrotoxicity and hepatotoxicity. Patients are monitored carefully for signs of rejection, infection, loss of allograft function, and adverse events associated with medications in the early posttransplant period. Patients on chronic immunosuppressive therapy have an incidence of malignancy of 5 to 6%, or approximately 100 times greater than that in the general population of the same age range. The most common lesions are cancer of the skin and lips and carcinoma in situ of the cervix, as well as lymphomas, such as non-Hodgkin's lymphomas. The risks are increased in proportion to the total immunosuppressive load administered and time elapsed since transplantation. Surveillance for skin and cervical cancers is necessary. Dose adjustments and drug substitutions may be indicated to stabilize graft function and decrease adverse events.

CsA and TAC are concentration-controlled. This means that the drugs are prescribed at a dose that produces a safe, effective range of exposure over the dosing period. The range changes over time, generally reflecting reduced dosing with increasing time following transplantation. The addition of other drugs to regimens based on CsA or TAC therapy may also change the prescribed therapeutic range. The clinical consequence is that patients must be monitored for exposure to these drugs over time. Clinic visits routinely consist of blood sampling to measure the Cmin.

MMF, AZA, and SRL are dose-controlled. A standard dose is administered every day. Exposure to these drugs is not monitored on a routine basis. However, dosing may be limited by adverse events, including diarrhea, myelosuppression and hyperlipidemia, for each of these drugs, respectively. Unfortunately, available agents have demonstrated little effect on the prevention of chronic allograft dysfunction, and in the case of kidney transplantation, chronic allograft nephropathy. Consequently, the focus of evaluation of immunosuppression is shifting beyond parameters of short-term efficacy and safety. The new goals of therapy are: prevention of the immune response (acute rejection, vascular remodeling), prevention of complications of immunodeficiency (opportunistic infection, malignancy), and minimization of drug-induced and other nonimmune toxicities.

Tolerance induction and chimerism are other approaches being explored for long-term allograft survival. Complete immune tolerance to the graft would require that no T-cell is activated by any graft antigen (donor HLA peptides) at any time post-implantation. Thus, all T-cells responding to graft antigens would have to be eliminated or shut down. Chimerism is an approach which attempts to chimerize the host immune system such that both host and donor antigens are recognized as self. One approach is to transplant host marginated donor hematopoietic cells to the recipient in the course of the operative procedure.

While having described the invention in general terms, the embodiments of the invention will be further disclosed in the following examples.

### Example 1

### Anti-Murine MCP-1 (JE) in a Cardiac Transplant Model

To test the potential effect of anti-MCP-1/CCL2 antibody on graft tissue remodeling and fibrosis in chronic allograft rejection, a murine cardiac transplant model was used. The anti-MCP-1 antibody used was an anti-JE antibody prepared by immunizing Sprague Dawley rats. This surrogate antibody, described in applicants co-pending patent application U.S. Ser. No. 11/170,453 is useful in mouse model systems.

In the murine cardiac transplant model, which is a fully allogeneic combination model, C57/B6 (H-2b) mice receive heterotopic heart transplant from C3H(H-2k) mice. The recipients are treated with a short course of anti-CD45RB (clone MB23G2, obtained from ATCC, antibody prepared by BioExpress, Inc., West Lebanon, N.H.) therapy to induce tolerance and prevent acute rejection (Ariyan, 2003 J. Immunol, 171:5673). In this model, grafts surviving long-term will develop chronic rejection features including: vasculitis, intimal lesions, fibrointimal thickening, smooth muscle cell expansion and luminal stenosis. The effect of treatment on tissue remodeling was assessed mainly by histological analysis of cardiac grafts. The histological analyses include hematoxylin-eosin (HE) staining, Masson's trichrome (MT) staining and Verhoeff's elastin staining of cardiac grafts.

### Anti-JE Alone Significantly Prolonged Allograft Survival, Indicating its Anti-Inflammatory Effects.

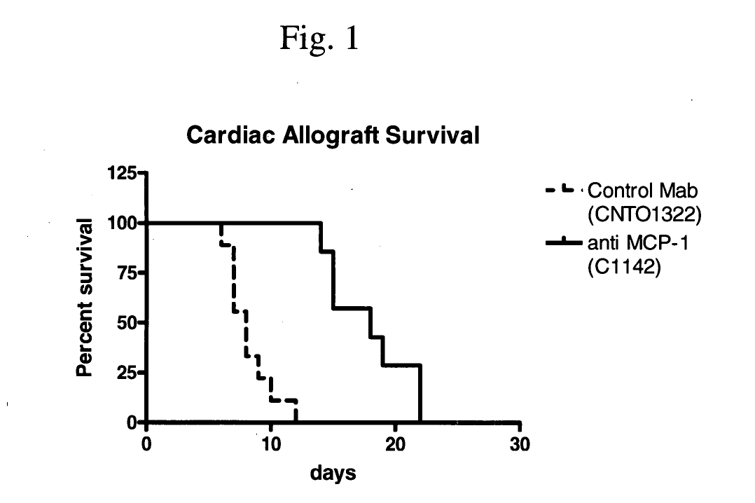

To test the impact of anti-JE on inflammatory immune responses, anti-JE was first tested on acute allograft rejection in the C3H to C57/B6 fully allogeneic cardiac transplant model. Recipients were treated with anti-JE or an irrelevant control mAb (1 mg/mouse i.p.) on days 0, 1, 3, 5 and 7, and heart graft function was monitored daily by direct abdominal palpation. The degree of function was scored as A, beating strongly; B, noticeable decline in the intensity of palpation; or C, complete cessation of cardiac impulses. Score A and B indicate graft survival. When cardiac impulses were no longer palpable, the graft was removed for routine histology. Statistical analysis of the survival data was performed using the T-test, and P values are two-tailed at 95% confidence. As shown in Table 1 and FIG. 1, graft survival was significantly prolonged in the anti-JE treated recipients. Anti-JE (antibody to the mouse homolog of MCP-1) significantly prolonged cardiac allograft survival. C57/B6 (H-2b) mice received heterotopic heart transplant from C3H(H-2k) mice, and the recipients were treated with anti-JE or an irrelevant control mAb (1 mg/mouse i.p.) on days 0, 1, 3, 5 and 7, and heart graft function was monitored daily by direct abdominal palpation.

TABLE 1

| Groups | N | Survival (day) | MST | P VALUES |
|---|---|---|---|---|
| Control (untreated) | 9 | 6, 7, 7, 7, 8, 8, 9, 10, 12 | 8.2 ± 1.9 | — |
| Control Ab | 5 | 7, 10, 11, 11, 13 | 10.4 ± 2.2 | P > 0.05 (vs Group 1) |
| Anti-JE Ab | 7 | 14, 15, 15, 18, 19, 22, 22. | 17.9 ± 3.3 | P < 0.05 (vs Group 1, 2) |

The present study indicates that anti-MCP treatment alone can prolong allograft survival, which provides insight in the effect of this mAb in preventing chronic graft rejection. These results indicate that murine MCP-1 homolog bioactivity, JE, is involved in the process of acute allograft rejection as blocking of the JE/chemokine receptor 2 (CCR2) signaling pathway with an anti-JE antibody has clear modulating effect on acute rejection.

### Example 2

### Anti-JE in Murine Model of Chronic Rejection

The impact of anti-JE on chronic graft rejection was tested in the allogeneic cardiac transplant model with the recipients receiving a short course of anti-CD45 RB. In this model, acute rejection of cardiac allografts is prevented by the treatment of anti-CD45RB mAb, but many grafts eventually undergo chronic rejection featured by vasculopathy and tissue remodeling. One month after the recipient mice were treated with a standard short course of anti-CD45RB, the animals were given either anti-JE or control Ab (100 microgram i.p. injection, twice weekly).

Heart graft function was monitored daily by direct abdominal palpation. Animals were terminated post 100 days of graft survival. At necropsy, tissue samples were removed, fixed in 10% buffered formaldehyde, and embedded in paraffin. Sections were cut with a microtome and stained with H/E and Trichrome staining. Specimens were examined microscopically and graded for severity of rejection by one lab pathologist and one investigator in a blinded fashion. More than five continuous sections were analyzed for each graft and criteria for chronic graft rejection included the presence of vasculitis, infarction, lymphocytic infiltration, thrombosis, intimal thickening, and hemorrhage. These changes were scored as 0 for no change, 1 for minimum change, 2 for mild change, 3 for moderate change, and 4 for marked change. As shown Table II, anti-JE treatment significantly suppressed arterial intimal thickening of the grafts, which is a hallmark of chronic rejection. In addition, anti-JE also showed a trend of inhibition of multiple parameters of chronic rejection including cellular infiltration, arteritis and fibrosis.

TABLE 2

| | | | Arterial | | |
|---|---|---|---|---|---|
| | Cell | | intimal | | |
| | infiltration | Arteritis | thickening | Fibrosis | Necrosis |
| Anti-CD45 | 3.2 ± 1.2 | 3.4 ± 0.8 | 3.2 ± 0.6 | 2.6 ± 1.2 | 2.2 ± 0.4 |
| only (n = 5) | | (66%) | (78%) | | |
| Anti-CD45 | ±1.0 | ±1.3 | ±0.4* | 2.2. + 1.6 | 1.6 ± 0.8 |
| plus anti- | | (45%) | (52%) | | |
| MCP-1/JE | | | | | |
| (n = 4) | | | | | |

Score grade: 0 = normal and 4 = most severe.
*P < 0.05

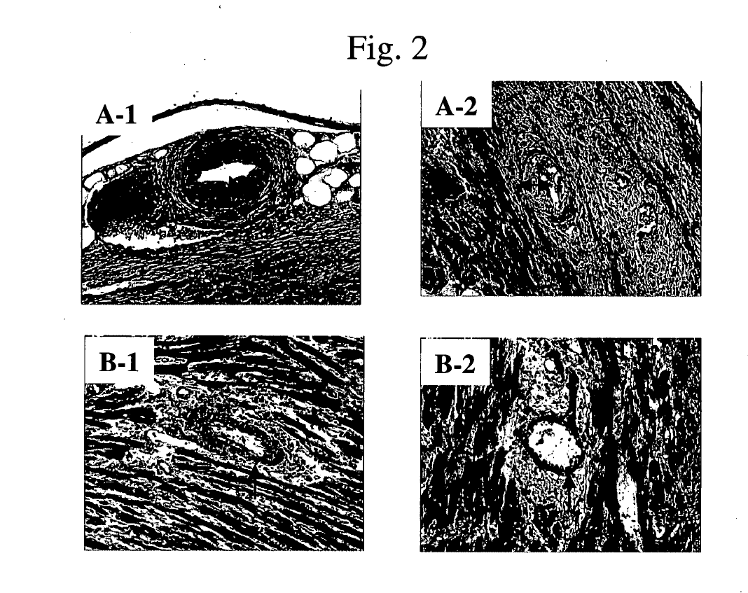

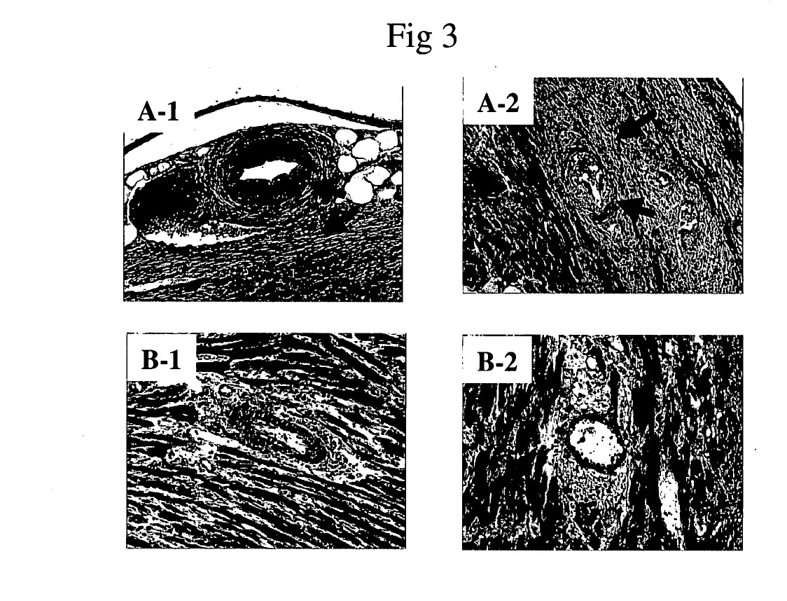

At necropsy, tissue samples were removed, fixed in 10% buffered formaldehyde, and embedded in paraffin. Sections were cut with a microtome and stained with Trichrome in order to detect collagen deposition. Specimens were examined microscopically in a blinded fashion. More than five continuous sections were analyzed for vascular intimal thickening. In FIGS. 2 and 3, A-1 and A-2 are two representative grafts from the anti-CD45RB treated group, while B-1 and B-2 from the anti-CD45RB plus anti-JE treated group, stained with H&E (FIG. 2) and using Trichrome (FIG. 3). These tissue sections show the presence of inflammatory cells, intimal thickening (indicated by black arrows) of the blood vessel wall in the H&E stain (FIG. 2a,b) and, in addition, collagen deposition was detected (FIG. 3a,b, black arrows) in the anti CD45RB only treated group, which are all hallmarks of chronic rejection. These features were notably absent or dramatically reduced in the groups treated with the combination of anti CD45RB and anti JE antibodies.

The key contribution of JE/MCP-1 in tissue remodeling associated with chronic graft rejection as indicated by the response to an anti-MCP-1 mAb indicates that anti-CCR2 antagonism could be an effective therapeutic approach to preventing, treating, and/or reversing chronic rejection related tissue remodeling.

For the first time, we have demonstrated that anti-JE (MCP-1) could prevent and control characteristic features of chronic rejection in allograft transplantation include vascular intimal thickening, arteritis and tissue fibrosis, and indicated that MCP-1 antagonists, including but not limited to mAb or small molecular drug to either MCP-1 or its receptor CCR2, could be a novel therapeutic for chronic rejection.

### REFERENCES

P. Hayry et al, Mechanisms of Chronic rejection, Transplantation Proceedings, 31(suppl 7A): 5S, 1999
A. M. Waaga et al, Mechanisms of chronic rejection, Current opinion in Immunology, 12: 517, 2000
A. K. Abbus et al, Chronic Rejection, Cellular and Molecular Immunology, 5th edition, P283, 2005
P. Libby and J. S. Pober, Chronic Rejection, Immunity, 14:387, 2001
E. M. Eugui, Fibrogenesis in chronic allograft rejection: Underlying mechanisms and pharmacological control, Transplantation Proceedings, 34: 2867, 2002
C. Daly et al, Monocyte chemoattractant protein-1 (CCL2) in inflammatory disease and adaptive immunity: therapeutic opportunities and controversies, Microcirculation, 3-4: 247, 2003
M Boratynska, The role of monocyte chemotatic peptide (MCP-1) in chronic renal allograft rejection, Pol Arch Med Wewn, 99: 272, 1998
K. C. Nadeau, et al, Sequential cytokine dynamics in chronic rejection of rat renal allografts: roles for cytokines RANTES and MCP-1, Proc Natl Acad Sci USA, 92: 8729, 1995
J. A. Belperio et al, Critical role for the chemokine MCP-1/CCR2 in the pathogenesis of bronchiolitis obliteras syndrome. J Clin Investig 108(4): 547-56, 2001.
C Ariyan et al, 2003. Transplantation tolerance through enhanced CTLA-4 expression. J. Immunol, 171:5673.

What is claimed:

1. A method for preventing or treating or reversing chronic rejection in a transplanted organ or tissue, which comprises administering a therapeutically effective amount of a medicament comprising an active agent capable of preventing CCL2 from binding to CCR2 to a mammalian recipient in need thereof.

2. The method of claim 1 wherein the method is for preventing chronic rejection.

3. The method of claim 2 wherein the transplantation is allograft transplantation.

4. The method of claim 3 wherein the allograft is a cardiac graft.

5. The method of claims 1 through 4 further comprising co-administering a therapeutically effective amount of an immunosuppressive agent.

6. The method of claim 1 wherein the active agent is an antibody.

7. The method of claim 6 wherein the antibody is an anti-MCP-1/CCL2 antibody.

8. The method of claim 7 wherein the medicament is for preventing chronic rejection.

9. The method of claim 8 wherein the transplantation is allograft transplantation.

10. A pharmaceutical composition for preventing and/or treating chronic rejection in a transplanted organ or tissue, which comprises a therapeutically effective amount of comprising an active agent capable of preventing CCL2 from binding to CCR2 in admixture with a pharmaceutically acceptable carrier or excipient.

11. The pharmaceutical composition of claim 10 wherein the composition is for preventing chronic rejection.

12. The pharmaceutical composition of claim 11 wherein the transplantation is allograft transplantation.

13. A method of prolonging graft survival in a patient using the pharmaceutical composition of claims 10 through 13, comprising co-administering a therapeutically effective amount of an immunosuppressive agent prior to, concurrently, or subsequently with the CCR2 antagonist.

14. The method of claim 13 wherein the immunosuppressive agent is a therapeutically effective combination of agent selected from the group consisting of cyclosporine, tacrolimus, sirolimus, mycophenolate mofetil, and prednisone.
